(19) United States
(12) Patent Application Publication
(10) Pub. No.: US 2026/0202373 A1
(43) Pub. Date: Jul. 16, 2026

(54) METHOD FOR MANUFACTURING AT LEAST ONE ELECTRODE OF AN ANALYTE SENSOR

(71) Applicant: Roche Diabetes Care, Inc., Indianapolis, IN (US)

(72) Inventors: Oleg Boguslawski, Mannheim (DE); Bernd Hiller, Mannheim (DE); Yilmaz Isgoeren, Mannheim (DE)

(73) Assignee: Roche Diabetes Care, Inc., Indianapolis, IN (US)

(21) Appl. No.: 19/563,310

(22) Filed: Mar. 11, 2026

Related U.S. Application Data
(63) Continuation of application No. 18/315,028, filed on May 10, 2023, now abandoned.
Application No. 18/315,028 is a continuation of application No. PCT/EP2021/081185, filed on Nov. 10, 2021.

(30) Foreign Application Priority Data
Nov. 12, 2020 (EP) 20207090.0

(51) Int. Cl. G01N 27/327; C12Q 1/00; H05K 3/12

(52) U.S. Cl. CPC G01N 27/327; C12Q 1/006; H05K 3/1225

(57) ABSTRACT

A method for manufacturing at least one electrode of an analyte sensor is disclosed. The method comprises the following steps, providing a stencil, wherein the stencil comprises a first stencil side, a second stencil side and at least one through hole reaching from the first stencil side to the second stencil side, wherein at least one of the first stencil side and the second stencil side has first wettability properties; providing a substrate, wherein the substrate comprises a first side and a second side; applying the stencil to the first side of the substrate; applying a low viscosity composition into the through hole of the stencil, wherein the low viscosity composition has second wettability properties opposing to the first wettability properties of the at least one of the first stencil side and the second stencil side; drying the low viscosity composition; obtaining the at least one electrode.

### TECHNICAL FIELD

The present invention relates to a method for manufacturing at least one electrode or test field of an analyte sensor and to an analyte sensor comprising the electrode or the test field, as well as to the use of the analyte sensor for detecting at least one analyte in a sample. The analyte sensor may, primarily, be used for a long-term monitoring of an analyte concentration in a bodily fluid, in particular of a glucose level or of a concentration of one or more other analytes in the bodily fluid. The invention may be applied both fields of home care and professional care, such as in hospitals. However, other applications are feasible.

### Background Art

Monitoring certain body functions, more particularly monitoring one or more concentrations of certain analytes, plays an important role in the prevention and treatment of various diseases. Without restricting further possible applications, the invention is described in the following with reference to glucose monitoring in an interstitial fluid. However, the invention can also be applied to other types of analytes. Glucose monitoring may, specifically, be performed by using electrochemical analyte sensors besides optical measurements. Examples of electrochemical analyte sensors for measuring glucose in body fluids are known from U.S. Pat. Nos. 5,413,690 A, 5,762,770 A, 5,798,031 A, 6,129,823 A or US 2005/0013731 A1.

In addition to “spot measurements” in which a sample of a bodily fluid is taken from a user, i.e. a human or an animal, in a targeted fashion and examined with respect to the analyte concentration, continuous measurements have become increasingly established. Thus, in the recent past, continuous measuring of glucose in the interstitial tissue, also referred to as “continuous glucose monitoring” or abbreviated to “CGM”, has been established as another important method for managing, monitoring, and controlling a diabetes state. Herein, an active sensor region is applied directly to a measurement site which is, generally, arranged in an interstitial tissue, and may, for example, convert glucose into an electrically charged entity by using an enzyme, in particular glucose oxidase (GOD) and/or glucose dehydrogenase (GDH). As a result, the detectable charge may be related to the glucose concentration and can, thus, be used as a measurement variable. Examples are described in U.S. Pat. No. 6,360,888 B1 or US 2008/0242962 A1.

During manufacturing of analyte sensors a chemical reagent needs to be applied on a sensor substrate, e.g. carbon, gold or plastic foils, accurate in position and shape. For high viscosity paste-like fluids such as manufactured on the basis of low volatile solvents, for example, screen-printing or rotary screen printing techniques can be used. However, discrete coating with water based or solvent-based low viscosity fluids is significantly more difficult. For example, applying thin lines having a line width of ≤4 mm, circles with a diameter of ≤4 mm, or squares or rectangles with a side length of ≤4 mm directly on the substrate may be very difficult. Screen-printing may not be possible since the fluid can flow on the surface of the sensor substrate and on the screen such that everything is wetted. As a standard technique, usually dispensing techniques are used, wherein single small droplets in single-digit nanolitre range may be applied to the substrate or lines may be applied on a moving substrate using a needle or cannula.

Despite the achievements of the above-mentioned techniques several challenges remain. Generally, a wetted surface of the substrate is defined by the surface tension of the fluid and the surface energy of the substrate. An application of sharp-edged rectangles may not be possible in this way. In addition, slow production speeds may occur for applying discrete two-dimensional elements such as circles. Moreover, coating of larger wet coating thicknesses of >50-100μm may not be possible because of bleeding of the fluid during drying. Even layered coating such as of electrode spots may be difficult to realize since the fluid may bleed into subjacent layers.

EP1690087 describes coated test elements, in particular test elements comprising a capillary gap. Said test elements comprise, at least in the area surrounding the capillary gap, a hydrophobically structured coating.

Pellitero et al. “Rapid prototyping of electrochemical lateral flow devices: stencilled electrodes”, Analyst 2016, 141, 2515 describes rapid prototyping of electrochemical lateral flow devices. It is proposed to prepare in site a stencil which may limit the available shapes and sized of the holes. Specifically, Pellitero et al. propose to glue on the liner, which will later serve as a template, and to apply pastes. The glue remains on the substrate under the paste. The stencil is hydrophobic on one side so that the glue that fixes the stencil to the substrate is not removed.

WO 2016/064881 A1 describes a paper substrate, microfluidic device which may be manufactured by using screen printing technology. The device includes a hydrophobic substrate to which a hydrophilic ink may be applied using a stencil. The document provides no details on the stencil.

WO 2014/025430 A2 describes methods, structures, devices and systems for fabricating electrochemical biosensors using a stencil.

KR 101 352 665 B discloses screen printed electrodes for biosensors and methods for manufacturing the same.

WO 2016/090189 A1 describes a non-invasive epidermal electrochemical sensor and a method for manufacturing the same.

### Problem to Be Solved

It is therefore an objective of the present invention to provide a method for manufacturing at least one electrode and/or at least one test field of an analyte sensor, an analyte sensor and a use thereof, which at least partially avoid the shortcomings of known analyte sensors and related methods and which at least partially address the above-mentioned challenges. Specifically, it is desired that the method and devices allow mass productional coating methods that enable coating of sharp edges and corners, in particular of 90 °, within small areas, such as of about <=3 mm.

### SUMMARY

This problem is addressed by a method for manufacturing at least one electrode of an analyte sensor, an analyte sensor and uses thereof with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a method for manufacturing at least one electrode of an analyte sensor is disclosed. Moreover, a method for manufacturing at least one test field of an analyte sensor is disclosed.

The methods are described in the following with reference to manufacturing the at least one electrode. However, as the skilled person immediately notices, the embodiments and definitions given can also be applied to the method for manufacturing the at least one test field.

The term “analyte sensor” as used herein is a broad term and is to be given its ordinary and customary meaning to a person of ordinary skill in the art and is not to be limited to a special or customized meaning. The term specifically may refer, without limitation, to an arbitrary device being configured to perform a detection of an analyte by acquiring at least one measurement signal for conducting at least one medical analysis. In particular, the analyte sensor may be an electrochemical sensor or an optical sensor.

As used herein, the term “electrochemical sensor” refers to an analyte sensor which is adapted for a detection of an electrochemically detectable property of the analyte, such as an electrochemical detection reaction. Thus, for example, the electrochemical detection reaction may be detected by applying and comparing one or more electrode potentials. Specifically, the electrochemical sensor may be adapted to generate the at least one measurement signal which may, directly or indirectly, indicate a presence and/or an extent of the electrochemical detection reaction, such as at least one current signal and/or at least one voltage signal. The measurement may be a qualitative and/or a quantitative measurement. Still, other embodiments are feasible.

As used herein, the term “optical sensor” refers to an analyte sensor which is adapted for performing at least one optical detection of the analyte. As used herein, the term “optical detection” refers to a detection of a reaction using an optical test chemical, such as a color-change test chemical which changes in color in the presence of the analyte. The color change specifically may depend on the amount of analyte present in the sample. Techniques for determining the analyte by optical detection and in particular analyzing color of a spot on the test field are generally known to the skilled person. Still, other embodiments are feasible. The term “test field” may relate to a continuous or discontinuous amount of test chemistry, also denoted test chemicals, which, preferably, is held by at least one carrier, such as by at least one carrier film, in the present case a substrate. The test chemistry may form or may be comprised in one or more films or layers of the test field, and/or the test field may comprise a layer setup having one or more layers, wherein at least one of the layers comprises the test chemistry. Thus, it is also envisaged by the present invention that the test chemistry comprises at least one chemical reagent for reacting with the analyte to produce the color change in the presence of the analyte. The test chemistry may be selected in respect to the analyte to be assessed. As is well known in the art, there are numerous chemistries available for use with each of various analytes. The selection of an appropriate chemistry is therefore well known within the skill in the art, and further description herein is not required in order to enable one to make and use the present invention.

The analyte sensor may be, in particular, an in vivo sensor. As particularly preferred, the analyte sensor may be a fully or partially implantable analyte sensor which may, particularly, be adapted for performing the detection of the analyte in a bodily fluid of a user in a subcutaneous tissue, particularly in an interstitial fluid. As used herein, the terms “implantable analyte sensor” or “transcutaneous analyte sensor” refers to an arbitrary analyte sensor being adapted to be fully or at least partly arranged within the body tissue of a patient or a user. For this purpose, the analyte sensor may comprise an insertable portion. Herein, the term “insertable portion” generally refers to a part or component of an element configured to be insertable into an arbitrary body tissue. Other parts or components of the analyte sensor may remain outside of the body tissue, e.g. counter electrode and/or reference electrode or combined counter/reference electrode may remain outside of the body tissue. Preferably, the insertable portion may fully or partially comprise a biocompatible surface, which may have as little detrimental effects on the user or the body tissue as possible, at least during typical durations of use. For this purpose, the insertable portion may be fully or partially covered with at least one biocompatibility membrane layer, such as at least one polymer membrane, for example a gel membrane.

Alternatively, the analyte sensor may be an ex vivo or in-vitro sensor. The analyte sensor may comprise at least one test element such as at least one electrochemical test element configured for detecting the analyte by using at least one electrochemical measurement, such as the measurement of at least one voltage and/or at least one current. Additionally or alternatively, other types of test elements may be used. The test element preferably is a test strip, i.e. a strip-shaped test element, such as a test element having a strip length of 5 mm to 100 mm, preferably 10 mm to 50 mm, and a strip width of preferably 1 mm to 30 mm, preferably 3 mm to 10 mm. The thickness of the test strips preferably is below 2 mm, preferably below 500 μm. The test strip preferably may be flexible such as deformable by hand. The test element may comprise the one or more chemical reagents, also denoted test chemicals, which, in presence of the analyte to be detected, are capable of performing one or more detectable detection reactions. With regard to the chemical reagents comprised in the test elements, reference may be made e.g. to J. Hoenes et al.: The Technology Behind Glucose Meters: Test Strips, Diabetes Technology & Therapeutics, Volume 10, Supplement 1, 2008, S-10 to S-26. Other types of chemical reagents are possible and may be used for performing the present invention.

As further generally used, both terms “user” and “patient” refer to a human being or an animal, independent from the fact that the human being or animal, respectively, may be in a healthy condition or may suffer from one or more diseases. As an example, the user or the patient may be a human being or an animal suffering from diabetes. However, additionally or alternatively, the invention may be applied to other types of users, patients or diseases.

As further used herein, the term “bodily fluid”, generally, refers to a fluid, in particular a liquid, which is typically present in a body or a body tissue of the user or the patient and/or may be produced by the body of the user or the patient. Preferably, the bodily fluid may be selected from the group consisting of blood and interstitial fluid. However, additionally or alternatively, one or more other types of bodily fluids may be used, such as saliva, tear fluid, urine or other body fluids. In case of an in vivo sensor, during the detection of the at least one analyte, the bodily fluid may be present within the body or body tissue. Thus, the analyte sensor may, specifically, be configured for detecting the at least one analyte within the body tissue.

As further used herein, the term “analyte” refers to an arbitrary element, component, or compound being present in the body fluid, wherein the presence and/or the concentration of the analyte may be of interest to the user, the patient, to medical staff, such as a medical doctor. In particular, the analyte may be or may comprise at least one arbitrary chemical substance or chemical compound which may participate in the metabolism of the user or the patient, such as at least one metabolite. As an example, the at least one analyte may be selected from the group consisting of glucose, cholesterol, triglycerides, lactate, in particular glucose. Additionally or alternatively, however, other types of analytes may be used and/or any combination of analytes may be determined. The determining of the at least one analyte specifically may, in particular, be an analyte-specific detection. Without restricting further possible applications, the present invention is described herein with particular reference to detecting and/or monitoring of glucose, in particular in an interstitial fluid.

The analyte sensor may comprise at least one electrochemical cell comprising at least one pair of electrodes. Specifically, the analyte sensor may comprise at least one working electrode and at least one further electrode and respective circuitry. The further electrode may be a counter electrode and/or a reference electrode or a combined counter/reference electrode. The working electrode may be sensitive for the analyte of interest at a polarization voltage which may be applied between working and reference electrodes and which may be regulated by a potentiostat. A measurement signal may be provided as an electric current between the counter electrode and the working electrode. A separate counter electrode may be absent and a pseudo reference electrode may be present, which may also work as a counter electrode.

The term “electrode” as used herein, is a broad term and is to be given its ordinary and customary meaning to a person of ordinary skill in the art and is not to be limited to a special or customized meaning. The term specifically may refer, without limitation, to an entity of the analyte sensor configured for contacting the bodily fluid, either directly or via at least one semi-permeable membrane or layer. The electrode may be embodied in a fashion that an electrochemical reaction may occur at at least one surface of the electrode. In particular, the electrode may be embodied in a manner that oxidative processes and/or reductive processes may take place at selected surfaces of the electrodes. Generally, the term “oxidative process” refers to a first chemical or biochemical reaction during which an electron is released from a first substance, such an atom, an ion, or a molecule, which is oxidized thereby. A further chemical or biochemical reaction by which a further substance may accept the released electron is, generally, denominated by the term “reductive process”. Together, the first reaction and the further reaction may also be denominated as a “redox reaction”. As a result, an electrical current, which relates to moving electrical charges, may be generated hereby. Herein, a detailed course of the redox reaction may be influenced by an application of an electrical potential.

The electrode may comprise a conductive material, in particular an electrically conductive material. As generally used, the term “conductive material” refers to a substance which is designed for conducting an electrical current through the substance. For this purpose, a highly conductive material having a low electrical resistance is preferred, in particular to avoid a dissipation of electrical energy carried by the electrical current within the substance. Preferably, the conductive material may be selected from a noble metal, especially gold; or from an electrically conductive carbon material; however, further kinds of conductive materials may also be feasible.

The electrode, in particular the working electrode, further may comprise the at least one chemical reagent disposed on the electrically conductive material. The chemical reagent may be or may comprise at least a polymeric material, specifically at least a polymeric material and at least a metal containing complex. The metal containing complex may be selected from the group of transition metal element complexes, specifically the metal containing complex may be selected from osmium-complexes, ruthenium-complexes, vanadium-complexes, cobalt-complexes, and iron-complexes, such as ferrocenes, such as 2-aminoethylferrocene. Even more specifically, the chemical reagent may be a polymeric transition metal complex as described for example in WO 01/36660 A2, the content of which is included by reference. In particular, the chemical reagent may comprise a modified poly (vinylpyridine) backbone loaded with poly(bi-imidizyl) Os complexes covalently coupled through a bidentate linkage. The chemical reagent may further be described in Feldmann et al, Diabetes Technology & Therapeutics, 5 (5), 2003, 769-779, the content of which is included by reference.

The method for manufacturing the at least one electrode of the analyte sensor comprises the following steps:
a) providing a stencil, wherein the stencil comprises a first stencil side, a second stencil side and at least one through hole reaching from the first stencil side to the second stencil side, wherein at least one of the first stencil side and the second stencil side has first wettability properties;
b) providing a substrate, wherein the substrate comprises a first side and a second side;
c) applying the stencil to the first side of the substrate;
d) applying a low viscosity composition into the through hole of the stencil, wherein the low viscosity composition has second wettability properties opposing to the first wettability properties of the at least one of the first stencil side and the second stencil side;
e) drying the low viscosity composition;
f) obtaining the at least one electrode.

Herein, the indicated steps may, preferably, be performed in the given order, whereby, in particular, the order of steps a) and b) can be exchanged without altering the result of the method. Further, additional steps, whether described herein or not, may be performed, too.

Specifically, the present invention proposes a method for manufacturing the at least one electrode, in particular a working electrode of the analyte sensor, by stencil printing of working electrode fields. The method is in particular applicable for mass production of analyte sensors. Use of stencil printing may allow manufacturing sharp edges and sharp 90° corners within small areas of about <=3 mm which is not possible with other mass production coating methods.

The term “stencil” as used herein is a broad term and is to be given its ordinary and customary meaning to a person of ordinary skill in the art and is not to be limited to a special or customized meaning. The term specifically may refer, without limitation, to at least one tool, in particular at least one template and/or at least one pattern and/or at least one mask, used for printing the composition onto the substrate. The stencil may define geometry and/or shape of the electrode to be manufactured. The term “geometry” may refer to dimensions of the electrode such as size, length, thickness and the like.

The stencil may comprise at least one foil having the at least one through hole. For example, the foil may be a metal foil or a plastic foil. Generally, arbitrary flexible foils can be used as stencil which are hydrophobic on both sides. In particular, foils having low surface energy, specifically, a low polar fraction of surface energy, may be used. For example, the polar fraction of the surface energy may be <10 mN/m, preferably <5 mN/m, wherein the polar fraction of the surface energy may be measurable by the Owens, Wendt, Rabel and Kaelble (OWKR)-method. Additional siliconization may be advantageous. In particular, if the material of the stencil may be magnetic, this may allow improved fixing of the stencil on the substrate. For example, the stencil may comprise at least one siliconized liner. Low surface energy may be realized by using a silicon or wax layer. Other kinds of foils may also be feasible. The term “providing” the stencil as used herein is a broad term and is to be given its ordinary and customary meaning to a person of ordinary skill in the art and is not to be limited to a special or customized meaning. The term specifically may refer, without limitation, to manufacturing the stencil and/or selecting a prefabricated stencil. As outlined above, the stencil comprises the at least one through hole. The providing of the stencil may comprise cutting and/or punching the through hole into the foil. The through hole may be cut into the foil by laser cutting and/or punching. The through hole may have the geometry and/or shape of the electrode to be manufactured. The stencil may comprise a plurality of through holes. The through holes may have diameters of ≤4 mm, preferably ≤1 mm, more preferably ≤0.5 mm. The stencil may have a pre-defined or selected thickness. The thickness of the stencil may define a wet film thickness, in particular of the low viscosity composition, later on during printing. For example, the stencil may have a thickness of ≥50 μm, preferably of ≥100 μm, more preferably ≥500 μm. The stencil may be provided, in particular manufactured, using a sheet-process and/or at least one roll-to-roll-process.

The stencil may have a planar shape. As generally used, the term “planar” refers to a body comprising extensions in two dimensions, typically denoted as “side” of the planar body, which exceed the extension in a third dimension, usually denoted as “thickness” of the planar body, by a factor of 2, at least a factor of 5, at least a factor of 10, or even at least a factor of 20 or more. The stencil may, specifically, have an elongated shape, such as a sheet or strip shape or a bar shape; however, other kinds of shapes may also be feasible. As generally used, the term “elongated shape” indicates that each side of the planar body has an extension in a direction along the elongation which exceeds an extension perpendicular hereto by at least a factor of 2, at least a factor of 5, at least a factor of 10, or even at least a factor of 20 or more. The extension perpendicular to the direction along the elongation may be denoted thickness.

The term “stencil side” as used herein is a broad term and is to be given its ordinary and customary meaning to a person of ordinary skill in the art and is not to be limited to a special or customized meaning. The term specifically may refer, without limitation, to an area of the stencil. The stencil may have four, in particular six, sides, two of which are opposite each other. The first stencil side may be the side of the stencil facing away from the substrate when the stencil is applied onto the substrate. The second stencil side may be the side of the stencil in contact with the substrate when the stencil is applied onto the substrate. A third and a fourth side, and in particular also a fifth and sixth side, of the stencil may define a height of the stencil. The orientation of the stencil may be pre-defined with respect to the substrate. However, embodiments may be possible wherein the first stencil side and the second stencil side are interchangeable, such that the stencil can be used in both orientations.

As used herein, generally, the terms “first”, “second” and “third” are considered as description without specifying an order and without excluding a possibility that other elements of that kind may be present.

The term “wettability property” as used herein is a broad term and is to be given its ordinary and customary meaning to a person of ordinary skill in the art and is not to be limited to a special or customized meaning. The term specifically may refer, without limitation, to surface wettability which relates, in particular, to surface free energy and geometric structures. Thus, in particular at least one of the surface of the first stencil side and the surface of the second stencil side has first wettability properties. The wettability property may be one or more of hydrophobic, hydrophilic, polar, or non-polar. The term “hydrophobic” as used herein is a broad term and is to be given its ordinary and customary meaning to a person of ordinary skill in the art and is not to be limited to a special or customized meaning. The term specifically may refer, without limitation, to a surface having a water contact angle of larger than 90°. The term “hydrophilic” as used herein is a broad term and is to be given its ordinary and customary meaning to a person of ordinary skill in the art and is not to be limited to a special or customized meaning. The term specifically may refer, without limitation, to a surface having a water contact angle of smaller than 90°. The contact angle may be measurable by using a goniometer. Techniques for measuring contact angle are known to the skilled person.

The first wettability property may be hydrophobic or hydrophilic. The second wettability property may be hydrophobic or hydrophilic. At least one of the first stencil side and the second stencil side may have opposing wettability properties than the low viscosity composition, in particular opposed polarities.

The term “opposing” within the context of the present invention and with regard to the wettability properties has the meaning as it is understood by the skilled person. In particular the term opposing means that the wettability has contrary behavior, for example with regard to the hydrophilicities and/or polarities. This means for example, that one wettability property is hydrophilic whereas the other wettability property is hydrophobic and/or one wettability property is polar and the other wettability property is non-polar (also called apolar). Usually, opposing wettability properties mean that the surface of the stencil and the low viscosity composition repel each other when they come into contact and/or they minimize their contact surface.

At least one of the first stencil side and the second stencil side may be hydrophobic and the low viscosity composition may be hydrophilic. Preferably both stencil sides may be hydrophobic. In particular, a whole or overall surface of the stencil may be hydrophobic. The term “whole or overall surface” of the stencil may refer to surfaces of the first stencil side, of the second stencil side and of holes. For example, the stencil may be hydrophobized with silicon. Alternatively, at least one of the first stencil side and the second stencil side may be hydrophilic and the low viscosity composition may be hydrophobic. Repulsive forces between the at least one stencil side and of the low viscosity composition have the effect that the low viscosity composition is fixed within the stencil, in particular the through hole, such that the substrate is not wetted over an area defined by the through hole. Reduced shearing of the low viscosity composition compared to dispensing cannulas with small diameters can be achieved.

In particular, the first stencil side and the second stencil side have low surface energy, specifically a low polar fraction of surface energy. This may allow that the low viscosity composition maintains within the through holes of the stencil and, does not spread above or below the stencil. Specifically, both of the first stencil side and the second stencil side may be hydrophobic. More specifically, both of the first stencil side and the second stencil side may have a low polar fraction of surface energy. This may allow that the, in particular waterbased, low viscosity composition, maintains a desired shape during a drying step.

A surface of the substrate, in particular the first side of the substrate facing the stencil, may have third wettability properties opposing to the first wettability properties. Specifically, the first side of the substrate may be hydrophilic or hydrophobic. In particular, the third wettability properties may be hydrophilic or hydrophobic.

The term “substrate” as used herein is a broad term and is to be given its ordinary and customary meaning to a person of ordinary skill in the art and is not to be limited to a special or customized meaning. The term specifically may refer, without limitation, to an arbitrary element designed to carry one or more other elements disposed thereon or therein. Particularly preferred, the substrate may be a planar substrate. The substrate may, specifically, have an elongated shape, such as a strip shape or a bar shape; however, other kinds of shapes may also be feasible. Specifically, the substrate may be a sheet. For example, the substrate may be provided as rolled-up sheet or tape. The substrate may be printed with the low viscosity composition and may be cut subsequently into the individual analyte sensors.

The substrate may comprise at least partially, preferably completely, at least one electrically insulating material, especially in order to avoid unwanted currents between electrically conducting elements as carried by the substrate. By way of example, the electrically insulating material may be selected from polyethylene terephthalate (PET) or polycarbonate (PC); however, other kinds of electrically insulating materials may also be feasible.

The substrate comprises the first side and a second side. As generally used, the term “side of the substrate” refers to an area of the substrate. In a particularly preferred arrangement, the first side and the second side of the substrate may constitute opposing sides of the substrate.

The first side of the substrate may comprise at least one conductive material. With respect to the term “conductive material” reference is made to the definition given above.

The providing of the substrate may comprise applying a layer of conductive material onto a side, in particular a first side of the electrically insulating material. The term “layer”, as used herein, refers to a volume comprising a material having extensions in two dimensions, typically denoted as “extension” of the layer, which exceed the extension in a third dimension, usually denoted as “thickness” of the layer, by a factor of 2, at least a factor of 5, at least a factor of 10, or even at least a factor of 20 or more, wherein the layer may be carried by the respective substrate, in particular, in order to provide stability and integrity to the layer. The layer may, specifically, have an elongated shape, such as a strip shape or a bar shape; however, other kinds of shapes may also be feasible. In general, the layer may, partially or completely, cover a respective side of substrate. In a preferred embodiment, in which the layer may only partially cover a portion of the respective side of the substrate, an insulating layer may, partially or completely, cover remaining portions of substrate. As used herein, the terms “apply” and “applying” the conductive material to the substrate refer to a process of depositing the conductive material on the substrate. In particular, the process may be selected from at least one of squeegee coating, chemical bath deposition, vacuum evaporation, sputtering, atomic layer deposition, chemical vapor deposition, spray pyrolysis, electrodeposition, anodization, electro-conversion, electro-less dip growth, successive ionic adsorption and reaction, molecular beam epitaxy, molecular vapor phase epitaxy, liquid phase epitaxy, inkjet printing, gravure printing, flexo printing, screen printing, stencil printing, slot die coating, doctor blading, and solution-gas interface techniques. For example, the substrate may be a carbon-coated substrate. The carbon-coated substrate may be manufactured using squeegee coating with purchased carbon pastes. For example, the substrate may be a gold-coated substrate. The substrate may comprise conductive layers on both sides. The second side of the substrate may, preferably, be blank, or may, as an alternative, comprise the at least one electrically insulating material. The term “lank”, as used herein, refers to an uncoated insulating surface.

As used herein, the terms “apply” and “applying” the stencil to the first side of substrate refer to a process of depositing the stencil on the first side of the substrate. The stencil may be applied to the substrate without using additional adhesive on the second stencil side. For example, the own weight of the stencil may be sufficient. Additionally or alternatively, the stencil may be fixed and/or weighed down on its outer edges. Additionally or alternatively, in case of a roll to roll process the stencil may be spanned over the substrate. The low viscosity composition may be prevented to flow under the stencil because of the hydrophobic and/or hydrophilic-interaction.

The term “composition” as used herein is a broad term and is to be given its ordinary and customary meaning to a person of ordinary skill in the art and is not to be limited to a special or customized meaning. The term specifically may refer, without limitation, to an arbitrary substance which comprises at least two different components, i.e. at least one first component and at least one second component. The low viscosity composition may comprise and/or may build and/or may form the chemical reagent of the electrode. Specifically, the low viscosity composition may comprise water and/or an osmium based polymer. The low viscosity composition may comprise reactive components for forming the chemical reagent. Other additives such as thickeners and/or surfactants may be omitted. Thus, in an embodiment, the low viscosity composition does not comprise a thickener and/or a surfactant.

The term “low viscosity” as used herein is a broad term and is to be given its ordinary and customary meaning to a person of ordinary skill in the art and is not to be limited to a special or customized meaning. The term specifically may refer, without limitation, to a property of the composition relating to low friction and high flow rate. Specifically, the low viscosity composition may be a fluid and/or a paste. The viscosity of the low viscosity composition may be ≤200 mPas, preferably ≤100 mPas, more preferably ≤50 mPas, preferably measured at 20° C. with a shear rate of 10 s⁻¹, preferably using a cone-plate viscometer. For example, the viscosity of the low viscosity composition may be about 50 mPas. For example, the viscosity of the low viscosity composition may be <100 mPas at 20° C. with a shear rate of 10 s⁻¹. The viscosity may be determined using a cone-plate viscometer. Such techniques are generally known to the skilled person.

The low viscosity composition is applied into the at least one through hole of the stencil. In particular, the low viscosity composition to be applied may be deposited on the stencil at an arbitrary position and may be one or more of spread, smeared, stripped over the stencil such as by using a squeegee or a wiper. By this process the at least one through hole may be filled with the low viscosity composition. High production speed may be possible, in particular by using arbitrary broad stencils with adapted spreading, smearing and stripping and/or by using roll-to-roll processes.

An application quantity per area of the low viscosity composition may be variable. The application quantity may be defined by the thickness of the stencil.

In step e), the method comprises at least one drying step in which the low viscosity composition is fully or partially dried. Thus, the low viscosity composition may be fixed during drying, in particular such that essentially no bleeding occurs. As used herein, the term “drying” generally refers to fully or partially removing one or more solvents from the low viscosity composition and/or by initiating one or more chemical reactions within the low viscosity composition, such as cross-linking reactions. In the latter case, the at least one chemical reaction may be initiated by internal factors, such as one or more initiators contained within the low viscosity composition, and/or may be initiated by one or more external influences, such as heat and/or electromagnetic radiation. The drying may be performed at room temperature or higher temperatures. For example, the drying may be performed at temperatures of ≤50° C. The drying may comprise one or more of: a heating; an exposure to hot gas, such as hot air; an exposure to electromagnetic radiation, preferably electromagnetic radiation in the ultraviolet spectral range. The drying may be performed, in particular, before removing the stencil from the substrate. However, embodiments in which the low viscosity composition is only partially dried, i.e. not fully cured or dried, before removing the stencil are feasible. In this case, an additional drying may be performed after removing the stencil.

Specifically, the low viscosity composition is only partially dried in the drying step. For example, in a batch process the stencil may be used several times or in a roll-to-toll process the stencil may be removed from the substrate as soon as possible. In these cases, the stencil may be removed from the substrate after reaching form stability in the drying step. The term “form stable” may refer to that the low viscosity composition maintains it shape or form without the stencil.

The method may comprise repeating step d), in particular for applying subsequently more than one layer of the low viscosity composition onto the substrate. Step d may be repeated before or after a drying step. In an embodiment when repeating step d) another low viscosity composition may be applied so that layered dots/squares etc. are obtained.

After the fully or partially drying of the low viscosity composition the stencil may be removed from the substrate. The term “obtaining the electrode”, as used herein, is a broad term and is to be given its ordinary and customary meaning to a person of ordinary skill in the art and is not to be limited to a special or customized meaning. The term specifically may refer, without limitation, to a process of completing the manufacturing of the electrode. The obtaining may comprise final manufacturing steps. As outlined above, the obtaining of the electrode may comprise removing the stencil from the substrate. In case, in step e), the low viscosity composition was only partially dried, step f) may comprise additional drying to finish the drying. The stencil may be removed during drying.

The proposed method for manufacturing the at least one electrode using a stencil with low surface energy, in particular a low polar fraction of surface energy may allow printing and/or coating of low viscosity compositions of arbitrary geometry on a substrate. Moreover, low viscosity compositions without rheological additives and/or surfactants can be used which in known methods have to be added to such a fluid for printing and/or coating.

In a further aspect a method for manufacturing at least one analyte sensor is disclosed. Generally, the method for manufacturing at least one analyte sensor comprises manufacturing at least one electrode according to one or more of the embodiments disclosed above and/or according to one or more of the embodiments disclosed in further detail below. Thus, for potential definitions, optional embodiments or other details of the method for manufacturing at least one analyte sensor reference may be made to the respective disclosure of the method for manufacturing at least one electrode.

The manufacturing of the analyte sensor may comprise cutting the substrate. Specifically, the manufacturing of the analyte sensor may comprise individualizing the analyte sensor. As further used herein, the term “individualize” generally refers to the process of separating the substrate into a plurality of analyte sensors. Thus, ready-to-use analyte sensors and/or intermediate products of the analyte sensors may be generated, which may optionally be subject to one or more subsequent finalization steps, such as coating steps. Those steps are known to the skilled person.

The manufacturing of the analyte sensor may comprise manufacturing at least one further electrode, in particular, in addition to the electrode. The further electrode may be manufactured to be arranged on an opposing side of the substrate than the electrode. The further electrode, preferably, may be manufacture before cutting the substrate.

In a further aspect, an analyte sensor for determining at least one analyte in a sample of bodily fluid is disclosed. Generally, the analyte sensor comprises at least one electrode manufactured by using a method for manufacturing at least one electrode according to one or more of the embodiments disclosed above and/or according to one or more of the embodiments disclosed in further detail below. Thus, for potential definitions, optional embodiments or other details of the analyte sensor reference may be made to the respective disclosure of the method for manufacturing at least one electrode.

Specifically, the analyte sensor is obtainable by
providing a stencil, wherein the stencil comprises a first stencil side, a second stencil side and at least one through hole reaching from the first stencil side to the second stencil side, wherein at least one of the first stencil side and the second stencil side has first wettability properties;
providing a substrate, wherein the substrate comprises a first side and a second side;
applying the stencil to the first side of the substrate;
applying a low viscosity composition into the through hole of the stencil, wherein the low viscosity composition has second wettability properties opposing to the first wettability properties of the at least one of the first stencil side and the second stencil side;
drying the low viscosity composition;
obtaining the at least one electrode;
cutting the substrate.

The obtaining of the at least one electrode may comprise manufacturing at least one further electrode, in particular, in addition to the electrode. The further electrode may be manufactured to be arranged on an opposing side of the substrate than the electrode. For example, the electrode may be a working electrode and the further electrode may be a reference and/or counter electrode. The further electrode, preferably, may be manufacture before cutting the substrate.

In a further aspect, a use of the analyte sensor for detecting at least one analyte in a sample is disclosed. For potential definitions, optional embodiments or other details of the use reference may be made to the respective disclosure of the method for manufacturing at least one electrode.

Summarizing and without excluding further possible embodiments, the following embodiments may be envisaged:

Embodiment 1. A method for manufacturing at least one electrode of an analyte sensor, the method comprising the following steps:
a) providing a stencil, wherein the stencil comprises a first stencil side, a second stencil side and at least one through hole reaching from the first stencil side to the second stencil side, wherein at least one of the first stencil side and the second stencil side has first wettability properties;
b) providing a substrate, wherein the substrate comprises a first side and a second side;
c) applying the stencil to the first side of the substrate;
d) applying a low viscosity composition into the through hole of the stencil, wherein the low viscosity composition has second wettability properties opposing to the first wettability properties of the at least one of the first stencil side and the second stencil side;
e) drying the low viscosity composition;
f) obtaining the at least one electrode.

Embodiment 2. A method for manufacturing at least one test field of an analyte sensor, the method comprising the following steps:
i. providing a stencil, wherein the stencil comprises a first stencil side, a second stencil side and at least one through hole reaching from the first stencil side to the second stencil side, wherein at least one of the first stencil side and the second stencil side has first wettability properties;
ii. providing a substrate, wherein the substrate comprises a first side and a second side;
iii. applying the stencil to the first side of the substrate;
iv. applying a low viscosity composition into the through hole of the stencil, wherein the low viscosity composition has second wettability properties opposing to the first wettability properties of the at least one of the first stencil side and the second stencil side;
v. drying the low viscosity composition;
vi. obtaining the at least one test field.

Embodiment 3. The method according to embodiment 1 or 2, wherein the first wettability properties are hydrophobic or hydrophilic, wherein the second wettability properties are hydrophobic or hydrophilic.

Embodiment 4. The method according to embodiment 1 or 2, wherein the first wettability properties are hydrophobic and the second wettability properties are hydrophilic or the first wettability properties are hydrophilic and the second wettability properties are hydrophobic.

Embodiment 5. The method according to any one of embodiments 1 to 4, wherein the second wettability properties are hydrophilic, wherein both stencil sides have the same first wettability properties, wherein the first wettability properties are hydrophobic.

Embodiment 6. The method according to any one of embodiments 1 to 5, wherein a whole surface of the stencil has first wettability properties, wherein the first wettability properties are hydrophobic.

Embodiment 7. The method according to any one of embodiments 5 or 6, wherein the stencil is hydrophobized with silicon.

Embodiment 8. The method according to any one of embodiments 1 to 7, wherein the low viscosity composition comprises water and/or an osmium based polymer.

Embodiment 9. The method according to any one of embodiments 1 to 8, wherein the viscosity of the low viscosity composition is ≤200 mPas, preferably ≤100 mPas, more preferably ≤50 mPas.

Embodiment 10. The method according to any one of embodiments 1 to 9, wherein the first side of the substrate has third wettability properties opposing to the first wettability properties, wherein the third wettability properties are hydrophilic or hydrophobic.

Embodiment 11. The method according to any one of embodiments 1 to 10, wherein the stencil has a thickness of ≥50 μm, preferably of ≥100 μm, more preferably ≥500 μm.

Embodiment 12. The method according to any one of embodiments 1 to 11, wherein the stencil comprises a plurality of through holes, wherein the through holes have diameters of ≤4 mm, preferably ≤1 mm, more preferably ≤0.5 mm.

Embodiment 13. The method according to any one of embodiments 1 or 3 to 12, wherein the first side of the substrate comprises at least one conductive material.

Embodiment 14. The method according to any one of embodiments 1 or 3 to 13, wherein the obtaining of the electrode comprises removing the stencil from the substrate.

Embodiment 15. The method according to any one of embodiments 2 to 12, wherein the obtaining of the test field comprises removing the stencil from the substrate.

Embodiment 16. A method for manufacturing at least one analyte sensor, the method comprising manufacturing at least one electrode according to the method of any one of embodiments 1 to 14, wherein the method further comprises obtaining the analyte sensor by cutting the substrate.

Embodiment 17. Analyte sensor for determining at least one analyte in a sample of bodily fluid, wherein the analyte sensor comprises at least one electrode manufactured by using a method according to any one of embodiments 1 or 3 to 14.

Embodiment 18. Analyte sensor for determining at least one analyte in a sample of bodily fluid, wherein the analyte sensor comprises at least one test field manufactured by using a method according to any one of embodiments 2 to 12 or 15.

Embodiment 19. The analyte sensor according to embodiment 17, wherein the analyte sensor is obtainable by
providing a stencil, wherein the stencil comprises a first stencil side, a second stencil side and at least one through hole reaching from the first stencil side to the second stencil side, wherein at least one of the first stencil side and the second stencil side has first wettability properties;
providing a substrate, wherein the substrate comprises a first side and a second side;
applying the stencil to the first side of the substrate;
applying a low viscosity composition into the through hole of the stencil, wherein the low viscosity composition has second wettability properties opposing to the first wettability properties of the at least one of the first stencil side and the second stencil side;
drying the low viscosity composition;
obtaining the at least one electrode;
cutting the substrate.

Embodiment 20. The analyte sensor according to embodiment 18, wherein the analyte sensor is obtainable by
providing a stencil, wherein the stencil comprises a first stencil side, a second stencil side and at least one through hole reaching from the first stencil side to the second stencil side, wherein at least one of the first stencil side and the second stencil side has first wettability properties;
providing a substrate, wherein the substrate comprises a first side and a second side;
applying the stencil to the first side of the substrate;
applying a low viscosity composition into the through hole of the stencil, wherein the low viscosity composition has second wettability properties opposing to the first wettability properties of the at least one of the first stencil side and the second stencil side;
drying the low viscosity composition;
obtaining the at least one test field;
cutting the substrate.

Embodiment 21. Use of the analyte sensor according to any one of embodiments 17 to 20 for detecting at least one analyte in a sample.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1A to 1F show a flow chart of a method for manufacturing at least one electrode of an analyte sensor according to the present invention (FIG. 1A and an example of a roll to roll process (FIG. 1B to 1F);

FIGS. 2A and 2B show an embodiment of step d) of the method;

FIGS. 3A to 3D show experimental results; and

FIG. 4 shows an embodiment of manufacturing a test strip using a method for manufacturing at least one analyte sensor according to the present invention.

### DETAILED DESCRIPTION OF THE EMBODIMENTS

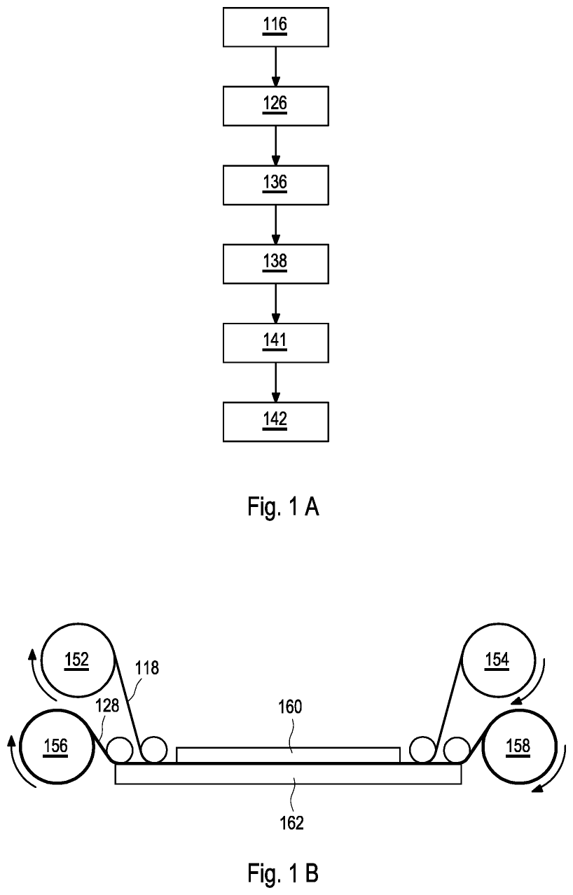

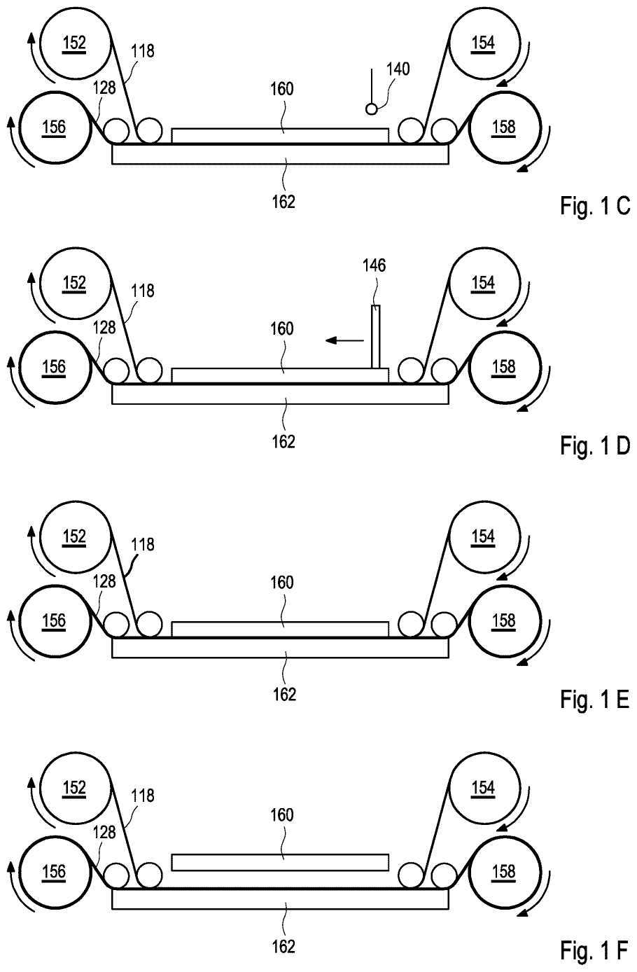

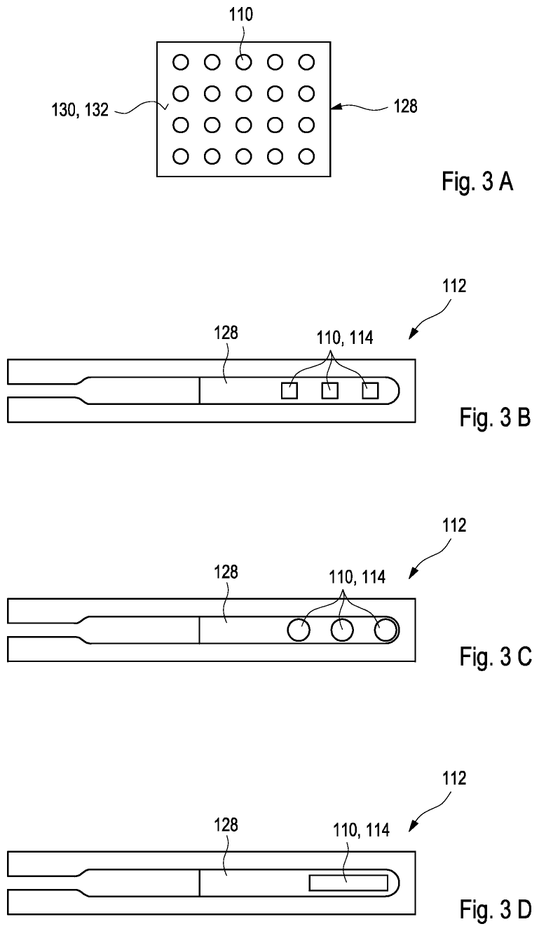

FIG. 1A shows a flow chart of a method for manufacturing at least one electrode 110 of an analyte sensor 112 according to the present invention. The electrode 110 and the analyte sensor 112 are shown in FIG. 3, for example.

The analyte sensor 112 may be configured to perform a detection of an analyte by acquiring at least one measurement signal for conducting at least one medical analysis. In particular, the analyte sensor 112 may be an electrochemical sensor. Specifically, the electrochemical sensor may be adapted to generate the at least one measurement signal which may, directly or indirectly, indicate a presence and/or an extent of the electrochemical detection reaction, such as at least one current signal and/or at least one voltage signal. The measurement may be a qualitative and/or a quantitative measurement. Still, other embodiments are feasible.

The analyte sensor 112 may be, in particular, an in vivo sensor. As particularly preferred, the analyte sensor 112 may be a fully or partially implantable analyte sensor 112 which may, particularly, be adapted for performing the detection of the analyte in a bodily fluid of a user in a subcutaneous tissue, particularly in an interstitial fluid. The analyte sensor 112 may be adapted to be fully or at least partly arranged within the body tissue of a patient or a user. For this purpose, the analyte sensor 112 may comprise an insertable portion. Other parts or components of the analyte sensor 112 may remain outside of the body tissue, e.g. counter electrode and/or reference electrode or combined counter/reference electrode may remain outside of the body tissue. Preferably, all electrodes of the implantable analyte sensor may be arranged at the end of a body of the analyte sensor. Preferably, the insertable portion may fully or partially comprise a biocompatible surface, which may have as little detrimental effects on the user or the body tissue as possible, at least during typical durations of use. For this purpose, the insertable portion may be fully or partially covered with at least one biocompatibility membrane layer, such as at least one polymer membrane, for example a gel membrane.

Alternatively, the analyte sensor 112 may be an ex vivo or in-vitro sensor. The analyte sensor 112 may comprise at least one test element such as at least one electrochemical test element configured for detecting the analyte by using at least one electrochemical measurement, such as the measurement of at least one voltage and/or at least one current. Additionally or alternatively, other types of test elements may be used. The test element preferably is a test strip, i.e. a strip-shaped test element, such as a test element having a strip length of 5 mm to 100 mm, preferably 10 mm to 50 mm, and a strip width of preferably 1 mm to 30 mm, preferably 3 mm to 10 mm. The thickness of the test strips preferably is below 2 mm, preferably below 500 μm. The test strip preferably may be flexible such as deformable by hand. The test element may comprise one or more chemical reagents, also denoted test chemicals, which, in presence of the analyte to be detected, are capable of performing one or more detectable detection reactions. With regard to the chemical reagents comprised in the test elements, reference may be made e.g. to J. Hoenes et al.: The Technology Behind Glucose Meters: Test Strips, Diabetes Technology & Therapeutics, Volume 10, Supplement 1, 2008, S-10 to S-26. Other types of chemical reagents are possible and may be used for performing the present invention.

The bodily fluid may be a fluid, in particular a liquid, which is typically present in a body or a body tissue of the user or the patient and/or may be produced by the body of the user or the patient. Preferably, the bodily fluid may be selected from the group consisting of blood and interstitial fluid. However, additionally or alternatively, one or more other types of bodily fluids may be used, such as saliva, tear fluid, urine or other body fluids. In case of an in vivo sensor, during the detection of the at least one analyte, the bodily fluid may be present within the body or body tissue. Thus, the analyte sensor 112 may, specifically, be configured for detecting the at least one analyte within the body tissue.

The analyte may be an element, component, or compound being present in the body fluid, wherein the presence and/or the concentration of the analyte may be of interest to the user, the patient, to medical staff, such as a medical doctor. In particular, the analyte may be or may comprise at least one arbitrary chemical substance or chemical compound which may participate in the metabolism of the user or the patient, such as at least one metabolite. As an example, the at least one analyte may be selected from the group consisting of glucose, cholesterol, triglycerides, lactate, in particular glucose. Additionally or alternatively, however, other types of analytes may be used and/or any combination of analytes may be determined. The determining of the at least one analyte specifically may, in particular, be an analyte-specific detection. Without restricting further possible applications, the present invention is described herein with particular reference to detecting and/or monitoring of glucose, in particular in an interstitial fluid.

The analyte sensor 112 may comprise at least one electrochemical cell comprising at least one pair of electrodes. Specifically, the analyte sensor 112 may comprise at least one working electrode 114, shown e.g. in FIGS. 3B to 3D, and at least one further electrode and respective circuitry. The further electrode may be a counter electrode and/or a reference electrode or a combined counter/reference electrode. The working electrode 114 may be sensitive for the analyte of interest at a polarization voltage which may be applied between working electrode 114 and reference electrode and which may be regulated by a potentiostat. A measurement signal may be provided as an electric current between the counter electrode and the working electrode 114. A separate counter electrode may be absent and a pseudo reference electrode may be present, which may also work as a counter electrode.

The electrode 110 may be an entity of the analyte sensor configured for contacting the bodily fluid, either directly or via at least one semi-permeable membrane or layer. The electrode 110 may be embodied in a fashion that an electrochemical reaction may occur at at least one surface of the electrode 110. In particular, the electrode 110 may be embodied in a manner that oxidative processes and/or reductive processes may take place at selected surfaces of the electrodes.

The electrode 110 may comprise an electrically conductive material. The electrically conductive material may be or may comprise a substance which is designed for conducting an electrical current through the substance. For this purpose, a highly conductive material having a low electrical resistance is preferred, in particular to avoid a dissipation of electrical energy carried by the electrical current within the substance. Preferably, the electrically conductive material may be selected from a noble metal, especially gold; or from an electrically conductive carbon material; however, further kinds of conductive materials may also be feasible.

The electrode 110, in particular the working electrode 114, further may comprise the at least one chemical reagent disposed on the electrically conductive material. The chemical reagent may be or may comprise at least a polymeric material, specifically at least a polymeric material and at least a metal containing complex. The metal containing complex may be selected from the group of transition metal element complexes, specifically the metal containing complex may be selected from osmium-complexes, ruthenium-complexes, vanadium-complexes, cobalt-complexes, and iron-complexes, such as ferrocenes, such as 2-aminoethylferrocene. Even more specifically, the chemical reagent may be a polymeric transition metal complex as described for example in WO 01/36660 A2, the content of which is included by reference. In particular, the chemical reagent may comprise a modified poly (vinylpyridine) backbone loaded with poly(bi-imidizyl) Os complexes covalently coupled through a bidentate linkage. The chemical reagent may further be described in Feldmann et al, Diabetes Technology & Therapeutics, 5 (5), 2003, 769-779, the content of which is included by reference.

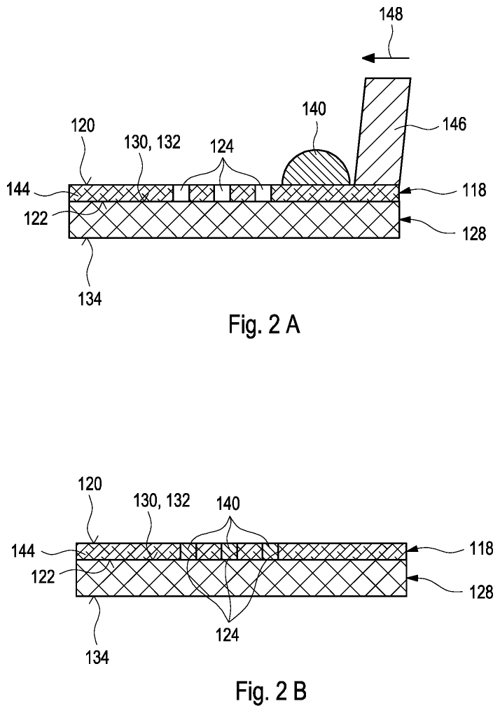

The method for manufacturing the at least one electrode 110 of the analyte sensor 112 comprises the following steps:
a) (denoted with reference number 116) providing a stencil 118, shown e.g. in FIG. 2, wherein the stencil 118 comprises a first stencil side 120, a second stencil side 122 and at least one through hole 124 reaching from the first stencil side 120 to the second stencil side 122, wherein at least one of the first stencil side 120 and the second stencil side 122 has first wettability properties;
b) (denoted with reference number 126) providing a substrate 128, shown e.g. in FIG. 2, wherein the substrate 128 comprises a first side 130 and a second side 134;
c) (denoted with reference number 136) applying the stencil 118 to the first side 130 of the substrate 128;
d) (denoted with reference number 138) applying a low viscosity composition 140 into the through hole 124 of the stencil 118, wherein the low viscosity composition 140 has second wettability properties opposing to the first wettability properties of the at least one of the first stencil side 120 and the second stencil side 122;
e) (denoted with reference number 141) drying the low viscosity composition 140;
f) (denoted with reference number 142) obtaining the at least one electrode 110.

Specifically, the present invention proposes a method for manufacturing the at least one electrode 110, in particular the working electrode 114, by stencil printing of working electrode fields. The method is in particular applicable for mass production of analyte sensors 112. Use of stencil printing may allow manufacturing sharp edges and sharp 90° corners within small areas of about <=3 mm which is not possible with other mass production coating methods.

The stencil, such as shown in FIGS. 2A and 2B, may be or may comprise at least one tool, in particular at least one template and/or at least one pattern and/or at least one mask, used for printing the composition onto the substrate 128. The stencil 118 may define geometry and/or shape of the electrode 110 to be manufactured.

The stencil 118 may comprise at least one foil 144 having the at least one through hole 124. For example, the foil may be a metal foil or a plastic foil. Generally, arbitrary flexible foils can be used as stencil which are hydrophobic on both sides. In particular, foils having low surface energy, specifically, a low polar fraction of surface energy, may be used. For example, the polar fraction of the surface energy may be <10 mN/m, preferably <5 mN/m, wherein the polar fraction of the surface energy may be measurable by the Owens, Wendt, Rabel and Kaelble (OWKR)-method. Additional siliconization may be advantageous. In particular, if the material of the stencil may be magnetic, this may allow improved fixing of the stencil on the substrate. For example, the stencil may comprise at least one siliconized liner. Other kinds of foils may also be feasible. The providing of the stencil 118 may comprise manufacturing the stencil 118 and/or selecting a prefabricated stencil 118. As outlined above, the stencil 118 comprises the at least one through hole 124. The providing of the stencil 118 may comprise cutting and/or punching the through hole into the foil 144. The through hole 124 may be cut into the foil 144 by laser cutting and/or punching. The through hole 124 may have the geometry and/or shape of the electrode to be manufactured. The stencil 118 may comprise a plurality of through holes 124. The through holes 124 may have diameters of ≤4 mm, preferably ≤1 mm, more preferably ≤0.5 mm. The stencil 118 may have a pre-defined or selected thickness. The thickness of the stencil 118 may define a wet film thickness later on during printing. For example, the stencil 118 may have a thickness of ≥50 μm, preferably of ≥100 μm, more preferably ≥500 μm. The stencil 118 may be provided, in particular manufactured, using a sheet-process and/or at least one roll-to-roll-process.

The stencil 118 may have a planar shape. The stencil 118 may, specifically, have an elongated shape, such as a sheet or strip shape or a bar shape; however, other kinds of shapes may also be feasible.

The stencil sides 120, 122 may be opposing, planar sides of the stencil 118. The first stencil side 120 may be the side of the stencil 118 facing away from the substrate 128 when the stencil 118 is applied onto the substrate 128. The second stencil side 122 may be the side of the stencil 118 in contact with the substrate 128 when the stencil 118 is applied onto the substrate 128. The orientation of the stencil 118 may be pre-defined with respect to the substrate 128. However, embodiments may be possible wherein the first stencil side 120 and the second stencil side 122 are interchangeable, such that the stencil 118 can be used in both orientations.

The wettability property may be surface wettability which relates, in particular, to surface free energy and geometric structures. The wettability property may be one or more of hydrophobic, hydrophilic, polar, or non-polar. At least one of the first stencil side 120 and the second stencil side 122 may have opposing wettability properties than the low viscosity composition 140, in particular opposed polarities. At least one of the first stencil side 120 and the second stencil side 122 may be hydrophobic and the low viscosity composition 140 may be hydrophilic. Preferably both stencil sides 120, 122 may be hydrophobic. In particular, a whole or overall surface of the stencil 118 may be hydrophobic. For example, the stencil 118 may be hydrophobized with silicon. Alternatively, at least one of the first stencil side 120 and the second stencil side 122 may be hydrophilic and the low viscosity composition 140 may be hydrophobic. Repulsive forces between the at least one stencil side 120, 122 and of the low viscosity composition 140 have the effect that the low viscosity composition 140 is fixed within the stencil 118, in particular the through hole 124, such that the substrate 128 is not wetted over an area defined by the through hole 124. Reduced shearing of the low viscosity composition 140 compared to dispensing cannulas with small diameters can be achieved.

A surface of the substrate 128, in particular the surface of the first side 130 facing the stencil 118, may have third wettability properties opposing to the first wettability properties. Specifically, said surface of the substrate 128 may be hydrophilic or hydrophobic.

The substrate 128 may be an arbitrary element designed to carry one or more other elements disposed thereon or therein. Particularly preferred, the substrate 128 may be a planar substrate. The substrate 128 may, specifically, have an elongated shape, such as a strip shape or a bar shape; however, other kinds of shapes may also be feasible. Specifically, the substrate 128 may be a sheet. For example, the substrate 128 may be provided as rolled-up sheet or tape. The substrate 128 may be printed with the low viscosity composition and may be cut subsequently into the individual analyte sensors.

The substrate 128 may comprise at least partially, preferably completely, at least one electrically insulating material, especially in order to avoid unwanted currents between electrically conducting elements as carried by the substrate. By way of example, the electrically insulating material may be selected from polyethylene terephthalate (PET) or polycarbonate (PC); however, other kinds of electrically insulating materials may also be feasible.

The first side of the substrate 128 may comprise at least one conductive material 132. The providing of the substrate 128 in step b) may comprise applying a layer of conductive material 132 onto a side, in particular a first side of the electrically insulating material. The layer may, specifically, have an elongated shape, such as a strip shape or a bar shape; however, other kinds of shapes may also be feasible. In general, the layer may, partially or completely, cover a respective side of substrate 128. In a preferred embodiment, in which the layer may only partially cover a portion of the respective side of the substrate 128, an insulating layer may, partially or completely, cover remaining portions of substrate 128. The applying of the layer of the conductive material 132 may comprise at least one process of depositing the conductive material 132 on the substrate 128. In particular, the process may be selected from at least one of squeegee coating, chemical bath deposition, vacuum evaporation, sputtering, atomic layer deposition, chemical vapor deposition, spray pyrolysis, electrodeposition, anodization, electro-conversion, electro-less dip growth, successive ionic adsorption and reaction, molecular beam epitaxy, molecular vapor phase epitaxy, liquid phase epitaxy, inkjet printing, gravure printing, flexo printing, screen printing, stencil printing, slot die coating, doctor blading, and solution-gas interface techniques. For example, the substrate may be a carbon-coated substrate. The carbon-coated substrate may be manufactured using squeegee coating with purchased carbon pastes. For example, the substrate may be a gold-coated substrate. The substrate may be conductive on both sides. The second side 134 of the substrate 128 may, preferably, be blank, or may, as an alternative, comprise the at least one electrically insulating material.

The applying of the stencil 118 on the substrate 128 in step c) may comprise depositing the stencil 118 on the first side 130 of the substrate 128. The stencil 118 may be applied to the substrate 128 without using additional adhesive on its second stencil side 122. For example, the own weight of the stencil 118 may be sufficient. Additionally or alternatively, the stencil 118 may be fixed and/or weighed down on its outer edges. Additionally or alternatively, in case of a roll to roll process the stencil 118 may be spanned over the substrate 128. The low viscosity composition may be prevented to flow under the stencil 118 because of the hydrophobic and/or hydrophilic-interaction.

The low viscosity composition 140 may be an arbitrary substance which comprises at least two different components, i.e. at least one first component and at least one second component. The low viscosity composition 140 may comprise and/or may build and/or may form the chemical reagent of the electrode. Specifically, the low viscosity composition 140 may comprise water and/or an osmium based polymer. The low viscosity composition 140 may comprise reactive components for forming the chemical reagent. Other additives such as thickeners and/or surfactants may be omitted. Thus, in an embodiment, the low viscosity composition does not comprise a thickener and/or a surfactant.

Specifically, the low viscosity composition 140 may be a fluid and/or a paste. The viscosity of the low viscosity composition may be ≤200 mPas, preferably ≤100 mPas, more preferably ≤50 mPas. For example, the viscosity of the low viscosity composition may be about 50 mPas. For example, the viscosity of the low viscosity composition may be <100 mPas at 20° C. with a shear rate of 10 s⁻¹. The viscosity may be determined using a cone-plate viscometer. Such techniques are generally known to the skilled person.

An embodiment of step d) of the method is shown in FIGS. 2A and 2B. The low viscosity composition 140 is applied into the at least one through hole 124 of the stencil 118. In particular, as shown in FIG. 2A, the low viscosity composition 140 to be applied may be deposited on the stencil 118 at an arbitrary position and may be one or more of spread, smeared, stripped over the stencil 118 such as by using a squeegee or a wiper 146. Movement of the squeegee or wiper 146 is visualized with arrow 148. As shown in FIG. 2B, by this process the at least one through hole 124 may be filled with the low viscosity composition 140. High production speed may be possible, in particular by using arbitrary broad stencils with adapted spreading, smearing and stripping and/or by using roll-to-roll processes.

An application quantity per area of the low viscosity composition 140 may be variable. The application quantity may be defined by the thickness of the stencil 118.

In step e), the method comprises at least one drying step in which the low viscosity composition 140 is fully or partially dried. Thus, the low viscosity composition 140 may be fixed during drying, in particular such that essentially no bleeding occurs. The drying in step e) may comprise fully or partially removing one or more solvents from the low viscosity composition 140 and/or by initiating one or more chemical reactions within the low viscosity composition 140, such as cross-linking reactions. In the latter case, the at least one chemical reaction may be initiated by internal factors, such as one or more initiators contained within the low viscosity composition 140, and/or may be initiated by one or more external influences, such as heat and/or electromagnetic radiation. The drying may be performed at room temperature or higher temperatures. For example, the drying may be performed at temperatures of ≤50° C. The drying may comprise one or more of: a heating; an exposure to hot gas, such as hot air; an exposure to electromagnetic radiation, preferably electromagnetic radiation in the ultraviolet spectral range. The drying may be performed, in particular, before removing the stencil 118 from the substrate 128. However, embodiments in which the low viscosity composition 140 is only partially dried, i.e. not fully cured or dried, before removing the stencil 118 are feasible. In this case, an additional drying may be performed after removing the stencil 118.

Specifically, the low viscosity composition is only partially dried in the drying step. For example, in a batch process the stencil 118 may be used several times or in a roll-to-toll process the stencil 118 may be removed from the substrate 128 as soon as possible. In these cases, the stencil 118 may be removed from the substrate after reaching form stability in the drying step.

The method may comprise repeating step d), in particular for applying subsequently more than one layer of the low viscosity composition 140 onto the substrate 128.

After the fully or partially drying of the low viscosity composition 140 the stencil 118 may be removed from the substrate 128. The obtaining the electrode 110 in step f) may comprise a process of completing the manufacturing of the electrode 110. The obtaining may comprise final manufacturing steps. As outlined above, the obtaining of the electrode 110 may comprise removing the stencil 118 from the substrate 128. The obtaining may comprise further steps such as cleaning the substrate 128. In case, in step e), the low viscosity composition was only partially dried, step f) may comprise additional drying to finish the drying. The stencil may be removed during drying.

FIGS. 1B to 1F show an exemplary embodiment of a roll-to-roll process of the method for manufacturing the at least one electrode 110 according to the present invention. The stencil 118, in a form of a liner, and the substrate 128 were provided as rolled-up sheet or tape. The liner may be unwinded from a first liner roll 152 and transported to a second liner roll 154 for winding. The substrate 128 may be unwinded from a first substrate roll 156 and transported to a second substrate roll 158 for winding. Liner and substrate may be positioned on top of each other. As shown in FIG. 1B, for the printing of the electrode 110 the transport may be stopped. A movable fixing frame 160 may be placed on top of the liner for weigh down the liner on a pressure table 162. FIG. 1C shows the step of applying the low viscosity composition 140 to the liner. The application may be done with a minimum surplus. In FIG. 1D shows spreading and/or smearing and/or stripping the low viscosity composition 140 over the liner such as by using the squeegee or the wiper 146. The squeegee or the wiper 146 may be moved over the liner with constant speed. FIG. 1E shows drying of the low viscosity composition 140. During drying the fixing frame 160 may be maintained on the liner. The drying may be performed at room temperature and/or by using nozzles. In FIG. 1F the fixing frame 160 is removed and substrate 128 and liner can be winded up.

FIGS. 3A to 3D shows experimental results. Specifically, in FIG. 3A a carbon substrate is shown with circular structures of low viscosity composition 140 each with a diameter of 0.8 mm applied using stencil printing. For this experiment, the low viscosity composition 140 was selected as water based polymer with a viscosity of around 50 mPas. The stencil 118 was manufactured by laser cutting. In particular a release liner was used which was hydrophobized with silicon on both sides. FIG. 3B shows an electrochemical analyte sensor 112 for continuous monitoring having three squared working electrodes 110, 114 being manufactured using the method according to the present invention on a carbon substrate. Each of the squares has a side length of 0.4 mm. FIG. 3C shows an electrochemical analyte sensor 112 for continuous monitoring having three circular working electrodes 110, 114 being manufactured using the method according to the present invention on a carbon substrate. Each of the circles has a diameter of 0.45 mm. FIG. 3D shows an electrochemical analyte sensor 112 for continuous monitoring having a rectangular working electrodes 110, 114 being manufactured using the method according to the present invention on a carbon substrate. The rectangle has a width of 0.45 mm and length of 2.00 mm.

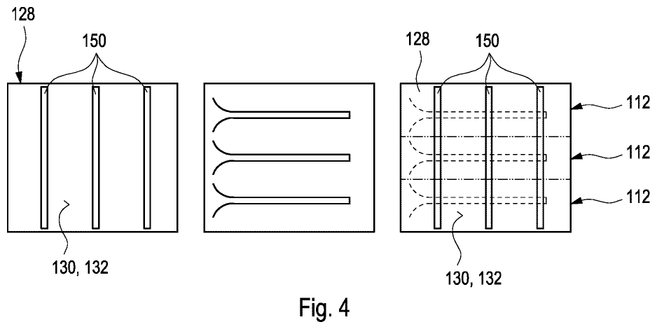

FIG. 4 shows an embodiment of manufacturing a test strip having a test field 150 using a method for manufacturing the at least one analyte sensor 112 according to the present invention. In particular, FIG. 4 shows coating of a transparent substrate 128, in particular a foil, with the low viscosity composition 140 for manufacturing capillary based diagnostic test strips comprising several reaction zones. From left to right in FIG. 4, in a first step, three different reagents were printed on the substrate 128 using the method for manufacturing at least one test field according to the present invention using stencil printing. In a second step, capillaries may be formed using a double-sided adhesive tape. In a third step, the printed substrate 128 and double-sided adhesive tape may be stuck together and cut.

### LIST OF REFERENCE NUMBERS

110 electrode
112 analyte sensor
114 working electrode
116 providing a stencil
118 stencil
120 first stencil side
122 second stencil side
124 through hole
126 providing a substrate
128 substrate
130 first side
132 conductive material
134 second side
136 applying the stencil
138 applying a low viscosity composition
140 low viscosity composition
141 drying
142 obtaining the electrode
144 foil
146 squeegee or wiper
148 arrow
150 test field
152 first liner roll
154 second liner roll
156 first substrate roll
158 second substrate roll
160 fixing frame
162 Pressure table

1. A method for manufacturing at least one electrode of an analyte sensor, the method comprising the following steps:
a) providing a stencil, wherein the stencil comprises a first stencil side, a second stencil side and at least one through hole reaching from the first stencil side to the second stencil side, wherein at least one of the first stencil side and the second stencil side has first wettability properties;
b) providing a substrate, wherein the substrate comprises a first side and a second side;
c) applying the stencil to the first side of the substrate;
d) applying a low viscosity composition into the through hole of the stencil, wherein the low viscosity composition has a viscosity of ≤200 mPas, measured at 20° C. with a shear rate of 10s−1 , and wherein the low viscosity composition has second wettability properties opposing to the first wettability properties of the at least one of the first stencil side and the second stencil side,
the first and second wettability properties being with regard to hydrophilicities and/or polarities;
e) drying the low viscosity composition;
f) obtaining the at least one electrode.

2. The method according to claim 1, wherein the first wettability properties are hydrophobic or hydrophilic, and wherein the second wettability properties are hydrophobic or hydrophilic.

3. The method according to claim 1, wherein the second wettability properties are hydrophilic, wherein both stencil sides have the same first wettability properties, wherein the first wettability properties are hydrophobic.

4. The method according to claim 1, wherein a whole surface of the stencil has first wettability properties, wherein the first wettability properties are hydrophobic.

5. The method according to claim 3, wherein the stencil is hydrophobized with silicon.

6. The method according to claim 1, wherein the low viscosity composition comprises water and/or an osmium based polymer.

7. The method according to claim 1, wherein the viscosity of the low viscosity composition is ≤200 mPas.

8. The method according to claim 1, wherein the first side of the substrate has third wettability properties opposing to the first wettability properties, wherein the third wettability properties are hydrophilic or hydrophobic.

9. The method according to claim 1, wherein the stencil has a thickness of ≥50 μm.

10. The method according to claim 1, wherein the stencil comprises a plurality of through holes, wherein the through holes have diameters of ≤4 mm.

11. The method according to claim 1, wherein the first side of the substrate comprises at least one conductive material.

12. The method according to claim 1, wherein the obtaining of the electrode comprises removing the stencil from the substrate.

13. A method for manufacturing at least one analyte sensor, the method comprising manufacturing at least one electrode according to the method of claim 1, wherein the method further comprises obtaining the analyte sensor by cutting the substrate.

14. Analyte sensor for determining at least one analyte in a sample of bodily fluid, wherein the analyte sensor comprises at least one electrode manufactured by using a method according to claim 1.

15. Use of the analyte sensor according to claim 14 for detecting at least one analyte in a sample.

16. The method according to claim 1, wherein one of the second stencil side or of the low viscosity composition is hydrophilic and the other is hydrophobic.

17. The method according to claim 1, wherein one of the second stencil side or of the low viscosity composition is polar and the other is apolar.

18. The method according to claim 7, wherein the viscosity of the low viscosity composition is ≤100 mPas.

19. The method according to claim 7, wherein the viscosity of the low viscosity composition is ≤50 mPas.

20. The method according to claim 9, wherein the stencil has a thickness of ≥100 μm.

21. The method according to claim 9, wherein the stencil has a thickness ≥500 μm.

22. The method according to claim 10, wherein the stencil comprises a plurality of through holes, wherein the through holes have diameters of ≤1 mm.

23. The method according to claim 10, wherein the stencil comprises a plurality of through holes, wherein the through holes have diameters of ≤0.5 mm.
